(19) United States
(12) Patent Application Publication
(10) Pub. No.: US 2023/0399605 A1
(43) Pub. Date: Dec. 14, 2023

(54) POROUS SCAFFOLD, METHOD OF MAKING AND USES THEREOF

(71) Applicant: University of Ottawa, Ottawa, Ontario (CA)

(72) Inventors: Jessica T. Holmes, Ottawa, Ontario (CA); Ziba Jaberansari, Ottawa, Ontario (CA); Wiliam Collins, Ottawa, Ontario (CA); Maxine Leblanc Latour, Ottawa, Ontario (CA); Daniel J. Modulevsky, Ottawa, Ontario (CA); Andrew E. Pelling, Ottawa, Ontario (CA)

(21) Appl. No.: 18/035,242

(22) PCT Filed: Nov. 9, 2021
(86) PCT No.: PCT/CA2021/051595

§ 371 Date: May 3, 2023

Related U.S. Application Data
(60) Provisional application No. 63/112,712, filed on Nov. 12, 2020.

(51) Int. Cl. C12N 5/00; A21D 13/40; A21D 2/02; A21D 2/26; A21D 2/14; A21D 8/04; A23L 13/00; A21D 8/06

(52) U.S. Cl. CPC C12N 5/0068; A21D 13/40; A21D 2/02; A21D 2/265; A21D 2/14; A21D 8/042; A23L 13/00; A21D 8/06; C12N 2537/10; C12N 2535/00; C12N 2533/90; C12N 2513/00

(57) ABSTRACT

The disclosure provides a bread-derived scaffold biomaterial for supporting cells, the scaffold comprising a bread crumb and wherein the bread crumb comprises a three-dimensional porous structure to support the cells. Further provided is a use of the scaffold and a method of making same.

### TECHNICAL FIELD

The present disclosure relates to a scaffold biomaterial to produce a tissue or other cell-based product.

### BACKGROUND

Plant-derived biomaterials have been reported for tissue engineering applications. In particular, previous work from the inventors demonstrates that the decellularization of plant tissues resulted in cellulose-rich three-dimensional (3D) scaffolds (WO2020/227835 and WO2017/136950). In addition, the inventors have demonstrated that these scaffolds also perform well after implantation into animal models, resulting in a high degree of tissue integration and vascularization. Other groups have reported similar findings with plant tissues and mammalian cell types to demonstrate the utility of plant-derived biomaterials for biomedical and food-based tissue engineering applications. However, such approaches are reliant on the structure and mechanical properties of the natural starting material.

Plant-derived proteins have been examined for creating supports for mammalian cells in tissue engineering applications. Proteins such as soy, zein and camelina have been studied, as well as gluten proteins derived from wheat, such as gliadin and glutenin. These wheat derived proteins can be purified and made into films suitable to culture mammalian cells. Reports have shown that such gluten films are acceptable substrates for osteoblasts. Further, a gluten film was shown to support the growth of osteoblasts but with less efficiency due to the cytotoxicity of gliadin. In another approach, wheat protein-based scaffolds have been prepared by electrospinning, in which ultrafine fibrous structures are obtained, creating a polymer melt film of wheat glutenin. Such scaffolds have been shown to support the culture of adipose derived mesenchymal stem cells. However, these methods are labor and resource intensive, requiring two days to purify the proteins and seven days for them to be electro-spun.

Beyond bio-medicine application, the development of naturally-derived 3D biomaterials has gained considerable interest in recent years due to their potential for use in the food industry [5,12,16]. Although still emerging, the broader goal of cellular agriculture is to replace products produced by traditional agricultural methods with biotechnological approaches, notably through synthetic biology and tissue engineering [17,18]. One specific area of interest within the field is the cultivation of mammalian cells in vitro for the preparation of meat-like products. Although a number of challenges remain to achieve this goal, a significant body of work has begun to address issues such as the large-scale production of relevant cell types, creating sustainable and ethically sourced media and developing suitable scaffolds. While exciting, it is important to also recognize that the future potential of these methods is still debated as efforts to address the continued dependence on animal products in traditional cell culture (for example, fetal bovine serum), water/electricity use and any potential health benefits in the final meat products are still ongoing. With that said, there remains an intense interest in developing solutions which address these problems, and others, potentially opening up a new future in which foods can be more sustainably created and distributed globally. Plant-derived scaffolds are of particular interest due to the potential of creating edible scaffolds. There is a need in the art for methods to prepare scaffolds for tissue culture which are simple and straightforward to implement.

Scaffolds containing starch and other materials such as ethylene vinyl alcohol have been fabricated for biomedical engineering applications. A primary area of interest is the use of starch blended with polymers or other biomaterials to serve as a bone tissue scaffold. Such scaffolds may be prepared by extrusion. (Salgado et al., 2004, Tissue Engineering, 10(3/4):465-474). Starch has been described as a polymer for wound healing applications. Investigators have fabricated starch-based nanofibrous scaffolds by electrospinning for wound healing applications. (Waghmare et al., 2018, Bioactive Materials, 3:255-266).

Fiume et al., 2019, Molecules, 24:2954 describes the use of bread as a template to make an inorganic scaffold. To prepare the inorganic scaffold, the bread was coated with a glass powder and then subjected to elevated temperature conditions to “burn off” the bread, leaving only the inorganic material comprising silica. The process described is limited to providing a material that served as an inorganic scaffold for bone engineering. Further, such process is reliant on a number of complicated steps to prepare the silica-containing scaffold.

The present disclosure provides one or more improvements and/or useful alternatives to providing scaffolds for producing a tissue or other cell-based product for use in a variety of applications.

### SUMMARY

The present disclosure relates to a bread-derived scaffold biomaterial to produce a tissue or other cell-based product for use in a variety of applications.

According to one aspect of the disclosure, there is provided a bread-derived scaffold biomaterial for supporting cells, the scaffold comprising a bread crumb and wherein the bread crumb has a three-dimensional porous structure to support the cells.

According to another aspect of the disclosure, there is provided a tissue or cell-based product having a three-dimensional structure comprising the bread-derived scaffold biomaterial, the bread-derived scaffold biomaterial having pores supporting a population of the cells.

According to a further embodiment, the tissue or cell-based product is a food product.

In one embodiment, the food product is a meat product. In such embodiment, the population of cells comprise myocytes and/or adipocytes.

According to another aspect of the disclosure, there is provided a process for producing a tissue or cell-based product comprising growing cells on the bread-derived scaffold biomaterial.

According to another aspect of the disclosure, there is provided a use of a bread-derived scaffold biomaterial to produce a tissue or a cell-based product.

In a further aspect, there is provided a method for producing a bread-derived scaffold biomaterial, comprising:
(i) preparing a dough comprising mixing a liquid, flour and a leavening agent;
(ii) baking the dough to produce a bread-product having an internal crumb; and
(iii) removing the internal crumb or a portion thereof from the bread-product to thereby provide the bread-derived scaffold biomaterial.

According to one embodiment of any of the foregoing aspects, the bread crumb is derived from a bread that is yeast-free.

According to any one of the foregoing aspects or the embodiment, the bread crumb is leavened with a bicarbonate salt, such as sodium bicarbonate.

According to any one of the foregoing aspects or embodiments, the crumb comprises one or more gluten proteins.

According to any one of the foregoing aspects or embodiments, the crumb comprises one or more non-gluten proteins.

According to any one of the foregoing aspects or embodiments, the bread crumb is cross-linked with a cross-linking agent.

According to any one of the foregoing aspects or embodiments, the cross-linking agent is glutaraldehyde or transglutaminase.

According to any one of the foregoing aspects or embodiments, the crumb is for supporting the growth of cells that are selected from mammalian cells, fish cells, avian cells, reptile cells, amphibian cells, crustacean cells, plant cells, invertebrate cells, algae cells, bacteria cells, archaea cells or fungal cells. In certain embodiments, the cell is a fibroblast, satellite cell, myoblast, myocyte, smooth muscle cell, myofibroblast, myotube, cardiomyocytes, neutrophil, macrophages, lymphocytes, monocytes, platelets, pre-osteoblast, osteoblast, osteoclast, pre-adipocyte, adipocyte, periodontal ligament stem cells, fibrocytes, chondrocyte, tenocyte, keratinocytes, hepatocytes, neuron, neural precursor cells, dorsal root ganglion cells, glial cells, astrocytes, epithelial cells, endothelial cells, stem, mesenchymal stem or induced pluripotent stem cell or any combination thereof.

According to any one of the above aspects or embodiments, the tissue includes skeletal muscle, smooth muscle, cardiac muscle, bone, fat/adipose, kidney, liver, lung, skin, neural, vascular tissues or any combination thereof.

According to any one of the foregoing aspects or embodiments, the cells are grown on the scaffold in vitro and/or in vivo.

In a further embodiment, the method described above further comprises a step of sterilizing the bread-derived scaffold biomaterial.

In a further embodiment, the method described above further comprises cross-linking the bread-derived scaffold biomaterial with a cross-linking agent.

In a further embodiment, the method described above comprises using a leavening agent that is a chemical agent. The chemical agent may be a salt of a bicarbonate ion.

### BRIEF DESCRIPTION OF DRAWINGS

FIG. 1 shows a method of producing baked bread (BB) scaffolds. The photos show A) Combining dry ingredients (flour, salt, baking powder). B) Assembling the ingredients with warm water to create a dough. C) After baking the dough, using the bread crumb is to prepare scaffolds for cell culture testing. D) Utilizing a 6 mm biopsy punch, preparing a 2.5 mm thick cylindrical scaffold from the bread crumb (scale bar=2 mm).

FIG. 2 shows a preliminary analysis of the BB scaffolds after incubation in cell culture media. Mechanical analysis of A) BB and B) cross-linked (glutaraldehyde) BB (xBB) scaffolds showing Young's Modulus (kPa) as a function of time in days. SEM imaging at Day 13 of C) BB and D) xBB scaffolds reveals their porosity is maintained over time under culture conditions (scale bar=1 mm and applies to both). Confocal imaging of congo red stained scaffolds also reveals the presence of pores in E) BB and F) xBB scaffolds (scale bar=400 μm and applies to both).

FIG. 3 shows Young's modulus of dry BB scaffolds (black), dry TG crosslinked scaffolds (tgBB, red) and tgBB scaffolds after incubation in culture conditions (blue) measured on a CellScale Univert at 2%/sec to a maximum of 85% strain (the Young's modulus was determined from fitting the linear regime, typically 10-30% compression).

FIG. 4 shows the characterization of BB and xBB scaffold microarchitectures. The scale bar=300 μm applies to all images as well as the depth scale. A) Confocal image of a BB scaffold where optical slices are coloured according to depth within the scaffold and maximum intensity Z-projected (color scale bar to the right of the image). B) Single optical section of the data in (A) 50 μm (left image) and 150 μm (right image) below the surface. C) A representative depth coded maximum intensity Z-projection image of an xBB scaffold is presented for comparison to the BB scaffold in (A). D) A representative confocal image of the BB scaffold. E) A representative confocal image of the xBB scaffold. F) Shows the range of pore sizes in diameter (μm) extracted from confocal images of BB (red) and xBB (black) scaffolds. G) Shows the volume fraction (%) of empty space for BB (left) and xBB (right) scaffolds.

FIG. 5 shows BB scaffolds (n=3) cut in half on Day 13 and a central region of the internal portion of the scaffold was then imaged. The A) scaffold; B) cell bodies; C) nuclei and D) merged image reveal that the cells are able to penetrate deeply inside of the scaffold (scale bar=300 μm and applies to all).

FIG. 6 shows a cross section of a typical xBB scaffold in which cells are observed deeper within the biomaterial. In this case, a cylindrical scaffold was cut in half to produce a half-cylinder. The internal portion of the scaffold was then imaged on the confocal microscope (scale bar=300 μm). In this example, NIH3T3 mouse fibroblasts stably expressing GFP were grown on the scaffold as described for 14 days before imaging.

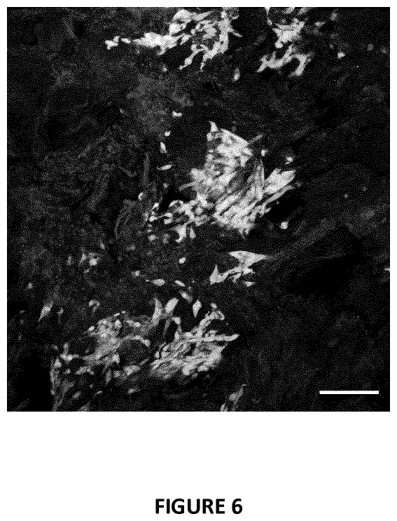

FIG. 7 shows microscopy and cell proliferation analysis of the BB and xBB scaffolds (scale bars=300 μm for Figs. A-F and 100 μm for Fig. G). A) Shows microscopy images of cell density on Day 2, after seeding, for BB scaffolds. B) Shows microscopy images of cell density on Day 2 after seeding for xBB scaffolds. C) Shows microscopy images of cell density on Day 13 after seeding for BB scaffolds. D) Shows microscopy images of cell density on Day 13 for xBB scaffolds. E) Higher magnification images of the BB scaffolds. F) Higher magnification images of the xBB scaffolds. G) SEM image of cells growing on a BB scaffold. H) Quantification of cell density on BB (red) and xBB (black) scaffolds.

FIG. 8 shows A) BB scaffolds containing B) NIH3T3-GFP cells after two weeks of culture that were stained for C) fibronectin. D) A merged image showing the scaffold stained with calcofluor white (blue), NIH3T3 cells (green) and fibronectin deposits (red) (scale bar=300 μm and applies to all).

FIG. 9 Culture and differentiation of C2C12 mouse myoblast cells on BB scaffolds. A) Mouse myoblasts after two weeks in culture (blue=nuclei, green=actin, red=scaffold). B) After differentiating the cells for two weeks they were observed to fuse into elongated multinucleated myotubes which appear green (blue=nuclei, green=myosin heavy chain, red=scaffold). Non-differentiated myoblasts are also visible as expected and appear as single blue nuclei which are not surrounded by any green staining. Scale bar=100 μm and applies to both images, both images are confocal maximum intensity Z-projections.

FIG. 10 Culture and differentiation of MC-3T3 mouse pre-osteoblast cells on BB scaffolds. A) A confocal maximum intensity Z-projection of MC-3T3 mouse pre-osteoblasts after two weeks in culture on a BB scaffold, scale bar=50 μm (blue=nuclei, green=actin, red=scaffold). B) A 3D reconstruction of a wider field of view of the data in (A) to show the 3D nature of the scaffold, scale bar=50 μm. C) Averaged EDS spectra of scaffolds containing cells cultured in osteogenic media (OM) (black), proliferation media (red) or a control scaffold with no cells (blue) (n=3 in each case). D) The Ca/P ratios for scaffolds of C) including scaffolds containing cells cultured in osteogenic media (OM), proliferation medium (BB) and the control scaffold with no cells (BB CTRL).

FIG. 11 shows metabolic data for BB and xBB bread scaffolds. A) an Alamar blue assay that reveals the relative fold-increase in metabolic activity of NIH3T3 fibroblasts after 1 day and 13/14 days of culture on the BB (red; leftmost dataset) and xBB (black; rightmost dataset) scaffolds. B) lactate dehydrogenase (LDH) and C) glutathione (GSH) assays for assessment of cytotoxicity and oxidative stress when cells are cultured on tissue culture plastic (TCP) and BB scaffolds.

### DETAILED DESCRIPTION

### Scaffold Biomaterial

Described herein is a scaffold biomaterial comprising a bread crumb that has a three-dimensional porous structure that has been shown to support the growth of a variety of cell types. Advantageously, the inventors have found that bread-derived scaffolds can be used as an alternative to synthetic or animal-derived scaffolds and may be used in a number of applications, such as, but not limited to biomedical engineering, cosmetics, agriculture, for preparing edible product, and other applications known to those of skill in the art.

By the term “crumb”, it is meant a portion of a bread that has a three-dimensional porous structure resulting from the use of a leavening agent during its production. Generally, the crumb is the internal portion of a bread product, such as a loaf. For example, for breads comprising a crust, the crumb portion is the internal part of the bread that excludes the crust.

By the term “cell-based product”, it is meant any product comprising a plurality of cells that are differentiated or undifferentiated, including a tissue, and that is produced either in vitro or in vivo. This includes, without limitation, any product for human or animal use in biomedical or food applications.

By the term “microcarrier”, it is meant a support for cells in any form in liquid tissue culture in a vessel.

The crumb is derived from any suitable type of bread that allows for the growth of cells. This includes bread derived from leavening a dough with a leavening agent that generates a gas, which is typically carbon dioxide. In some embodiments, the porous structure allows for the infiltration, growth and/or migration of cells within the crumb structure.

The pore sizes in the crumb can vary significantly over a range of micrometers to millimeters. The leavening agent and/or its concentration may be selected to provide a desired and/or consistent pore size to the scaffold. The pore size may be selected to optimize the growth and/or migration of the cells in the scaffold. In one embodiment, a high porosity scaffold is employed to avoid an anoxic environment within the scaffold structure.

A suitable pore size can be assessed by a variety of known techniques including image analysis. Scaffolds may be imaged by optical microscopy or by scanning electron microscopy (SEM). The pores in the scaffold may be partially or completely interconnected as determined by microscopy. A scaffold having suitable pore sizes and interconnectivity thereof can be readily selected by those of skill in the art. Assessment of pore size and interconnectivity can be carried out by techniques described in Ashworth and Cameron, 2014, Materials Technology, Advanced Performance Materials, 29(5):281-295, relevant sections being incorporated herein by reference.

### Preparation of the Scaffold Biomaterial

In some embodiments, the bread crumb for the scaffold is most advantageously prepared from a bread that is leavened without yeast. A non-limiting example of a suitable leavening agent is sodium bicarbonate, although other non-yeast leavening agents may be used as required. For example, the scaffold may be prepared from bread crumb derived from a soda bread. Soda bread is generally prepared with ingredients comprising flour, salt and sodium bicarbonate and does not contain biological cultures, such as yeast. However, the crumb may be prepared from other types of breads, including in some embodiments, those leavened with yeast or other biocatalysts.

By way of example, a soda bread for use in preparation of the crumb may be prepared using appropriate amounts of flour, salt and a leavening agent, such as baking power as dry ingredients, which are admixed together or separately with water. The resultant mixture may be kneaded and then baked at an appropriate temperature to facilitate rising. The crumb may be obtained from the baked product by removing an internal portion thereof using any mechanical implement suitable for such use. In one example, a biopsy punch is used, although other methods for removing the internal crumb portion may be utilized as would be appreciated by those of skill in the art.

In one non-limiting embodiment, the crumb is sterilized prior to its use as a scaffold. This may be carried out by any suitable method. For example, the crumb may be contacted with a sterilizing agent, such as an alcohol or other chemical that is capable of destroying or reducing the concentration of unwanted microorganisms in the crumb structure. For in vitro applications, to facilitate the growth of cells within the scaffold, the bread crumb may be treated with a liquid culturing media prior to seeding with cells to promote adherence thereof. The culture media may be exchanged as required during the culturing process.

In one embodiment, the bread crumb comprises one or more proteins. For example, the crumb may comprise one or more gluten proteins. Advantageously, a gluten protein may impart stability to the scaffold. In another embodiment, the scaffold may comprise one or more non-gluten proteins, examples of which include albumin and globulins. Such proteins have utility in the creation of biomaterials. The bread crumb will typically comprise starch as well.

In certain embodiments, the scaffold may be chemically modified to introduce cross-linking. An example of a non-limiting cross-linking agent is glutaraldehyde (GA), although other cross-linking agents are encompassed by the present disclosure. In another embodiment, the cross-linking agent is transglutaminase. A suitable cross-linking agent may be selected based on the particular application for which the scaffold is used.

### Preparation of a Scaffold Biomaterial Comprising Cells

The bread crumb scaffold is cultured under conditions effective to produce a scaffold that supports a desired population of cells.

The bread crumb scaffold may be used to support a variety of different types of cells, or combinations of such cells. This includes, but is not limited to mammalian cells, fish cells, avian cells, reptile cells, amphibian cells, crustacean cells, plant cells, invertebrate cells, algae cells, bacteria cells, archaea cells or fungal cells. In certain embodiments, the cell is a fibroblast, satellite cell, myoblast, myocyte, smooth muscle cell, myofibroblast, myotube, cardiomyocytes, neutrophil, macrophages, lymphocytes, monocytes, platelets, pre-osteoblast, osteoblast, osteoclast, pre-adipocyte, adipocyte, periodontal ligament stem cells, fibrocytes, chondrocyte, tenocyte, keratinocytes, hepatocytes, neuron, neural precursor cells, dorsal root ganglion cells, glial cells, astrocytes, epithelial cells, endothelial cells, stem, mesenchymal stem or induced pluripotent stem cell or any combination thereof.

Culturing of the bread crumb scaffold with cells comprises introducing cells to the scaffold under conditions that promote growth and proliferation of the cells. In some embodiments, prior to seeding the scaffold, the cells are cultured in vitro under suitable conditions known to those of skill in the art.

To prepare bread scaffolds for seeding, the scaffold may be sterilized. The sterilization includes any suitable technique. In one embodiment, the bread scaffold is placed in a sterilizing solution that reduces or eliminates the concentration of unwanted microbes. In one embodiment, the sterilizing solution comprises an alcohol, such as ethanol. Subsequent to sterilization, the scaffold may be rinsed with a suitable solution, such as but not limited to a buffer to remove a chemical used for sterilizing.

In one embodiment, bread scaffolds are most advantageously soaked in culture media prior to seeding with cells to encourage adherence of the cells to the scaffold structure. The culture media may include serum, such as fetal calf serum or horse serum. The serum content may vary from 0.5% to 20% depending on the type of cells being cultured.

In those embodiments in which the bread scaffold is soaked, the cells are typically added after the soaking. The scaffold which is seeded with cells may be incubated for any period of time at a temperature effective to allow the cells to adhere to the scaffold. In one embodiment, the culture media is exchanged during incubation. The cells may be maintained on scaffolds for any desired period of time to facilitate their adherence, growth and/or proliferation within the three-dimensional structure of the scaffold. In some embodiments, the seeding with cells is repeated after a period of time.

The crumb scaffold or portions thereof finds use in microcarrier culturing used in industrial applications. According to such embodiments, the crumb scaffold may be introduced to a vessel, such as a bioreactor, and may function in a similar manner as a “microcarrier”, which is a support matrix (e.g., beads or other matrices) used to facilitate dense cell growth thereon and improve the yield of the tissue or cell-based product during manufacture. Often the support matrix is maintained in suspension in a culture medium in a vessel as particles with stirring, although the bread scaffold may be present in the vessel in any suitable solid form. Microcarrier culturing has broad applicability but may be particularly suitable for cells that rely on adherence to a support (e.g., adherent cells). Many conventional microcarrier culturing processes use beads, which have the limitation of low surface area. The crumb scaffold described herein is porous, thereby increasing surface area for cell adherence and growth. Accordingly, in some embodiments, the crumb scaffold is introduced into a vessel and functions as a microcarrier to increase available surface area for cell growth during proliferation in suspension culture. In addition to improved surface area offered by the scaffold of the disclosure, micro-carrier-based processes are advantageous in that they allow for more precise cell growth control, reduced bioreactor volume (thereby reducing space otherwise used to accommodate large bioreactors in an operation) and/or decreased labour costs.

Microcarrier-based processes may be carried out in a variety of vessels, including but not limited to spinner flasks, rotating wall microgravity bioreactors or fluidized bed bioreactors. In one non-limiting embodiment, a stirred bioreactor is most suitable for microcarrier culturing.

### Uses for the Scaffold

The crumb scaffold is for use to produce any tissue or cell-based product for use in an in vitro or in vivo application. Non-limiting examples are set forth below.

Examples of tissue include skeletal muscle, smooth muscle, cardiac muscle, bone, fat/adipose, kidney, liver, lung, skin, neural, vascular tissues or any combination thereof.

In one non-limiting embodiment, the crumb scaffold is used as a scaffold to prepare products for human or animal consumption. An example of an edible product for human or animal consumption is a meat product produced by tissue engineering. The meat may be used for human consumption or for pet food. Other edible products for human or animal consumption are encompassed by the present disclosure. In one embodiment, the crumb is used to produce a vegetarian food product.

In those embodiments in which the edible product is a meat product, the crumb scaffold may comprise myocytes, including precursors thereof. A myocyte includes those cells typically found in muscle tissue, including smooth muscle cells, cardiac muscle cells, skeletal muscle cells and combinations thereof. The myocyte includes a mammalian, avian or fish myocyte. The myocyte may be a myocyte substitute, which is a cell that can differentiate into myocytes or muscle cells under suitable conditions. The scaffold may also comprise adipocytes, including precursors or substitutes thereof.

Further embodiments include use of the bread crumb scaffold in therapy and/or biomedical applications. This includes the use of an implantable scaffold for supporting cell growth, for promoting tissue regeneration, for promoting angiogenesis, for a tissue replacement procedure and/or as a structural implant for cosmetic surgery. Further embodiments encompass therapeutic treatment and/or cosmetic methods employing such scaffolds, as well as other applications which may include veterinary uses. In one embodiment, the tissue is a soft tissue.

Further specific therapeutic or biomedical applications include tissue regeneration including soft tissue repair, neuro-regeneration, skin reconstruction, artificial corneas and skeletal/cardiac muscle regeneration.

In certain embodiments, scaffold biomaterials as described herein may be used as a structural implant for repair or regeneration following spinal cord injury; as a structural implant for tissue replacement surgery and/or for tissue regeneration following surgery; as a structural implant for skin graft and/or skin regeneration surgery; as a structural implant for regeneration of blood vasculature in a target tissue or region; as a tissue replacement for skin, spinal cord, heart, muscle, nerve, blood vessel, or other damaged or malformed tissue; as a vitreous humour replacement (in hydrogel form); as an artificial bursae, wherein the scaffold biomaterial forms a sac-like structure containing scaffold biomaterial in hydrogel form; and/or as a structural implant for cosmetic surgery.

In another embodiment, the scaffold biomaterial described herein is for use as a microcarrier in a vessel, such as a bioreactor, in order to support the growth and adherence of cells. The use of scaffold biomaterial as a microcarrier has wide ranging applications in therapy, biomedical applications and in the food industry.

### EXAMPLES

### Materials and Methods

### 1. Bread Recipe and Fabrication

Soda bread was prepared by adding, in a ceramic bowl, 120 g of all purpose flour (Five Roses™), 2 g of iodized table salt (Windsor™) and 10 g of baking power (Kraft™) and mixing. Subsequently, 70 mL of water was added to the dry ingredients. The water was previously heated for 30 seconds in a microwave until its temperature was about 75 degrees Celsius. The mixture was combined to form a dough and shaped into a ball. The dough was kneaded for 3 minutes with the addition of flour as needed to reduce sticking. Once flattened into a circular disk with a height of approximately 2.5 cm, the dough was place in a glass bread pan lined with parchment paper. The dough was baked for 30 minutes at 205 degrees Celsius in a preheated oven. The cooled bread was stored in a resealable plastic bag (Ziploc™) at −20 degrees Celsius until use.

When ready for use, the bread was thawed to room temperature. A 6 mm biopsy punch was used to extract cylindrical shapes from the internal portion of the loaf (also referred to herein as the “crumb”). The cylinders were cut with a blade (Leica™) to form circular scaffolds, which were about 2.5 mm in thickness. Two formulations were tested: the native untreated scaffolds as well as a group of chemically crosslinked scaffolds. To crosslink the samples with glutaraldehyde (GA), an adapted approach was used for similar protein-based scaffolds (R. Hickey, A. E. Pelling, The rotation of mouse myoblast nuclei is dependent on substrate elasticity, Cytoskeleton. 74 (2017) 184-194 and Z. Al-Rekabi, A. E. Pelling, Cross talk between matrix elasticity and mechanical force regulates myoblast traction dynamics, Phys. Biol. 10 (2013) 066003, each of which is incorporated herein by reference). A 0.5% GA solution was prepared from a 50% electron microscopy grade glutaraldehyde stock (Sigma™), which was diluted with PBS (Fisher™). The scaffolds were incubated in the GA solution overnight in the fridge. Afterwards, the scaffolds were rinsed 3 times with PBS. To reduce any remaining traces of unreacted glutaraldehyde, the scaffolds were incubated in a 1 mg/mL NaBH₄ (Acros Organics™) solution on ice, made immediately before use. Once the formation of bubbles ceased, the samples were rinsed 3 times with PBS. In some cases, the bread scaffolds were also crosslinked with transglutaminase (TG; Modernist Pantry™). TG is a well-known enzyme that catalyzes protein crosslinking by forming covalent links between the carboxamide and amino groups of glycine and lysine respectively. TG was mixed with the dry ingredients at a concentration of 1% (w/w) in advance of baking.

### 2. Cell Culture

NIH3T3 mouse cells stably expressing GFP were used in this study (ATCC). Cells were cultured in high glucose Dulbecco's Modified Eagle medium (MDEM) (HyClone™), supplemented with 10% fetal bovine serum (HyClone™) and 1% penicillin/streptomycin (HyClone™) at 37 degrees Celsius and 5% CO2. The culture media was exchanged every second day and the cells were passaged at 70% confluence. To test the suitability of the scaffold to support the proliferation of other cell types, C2C12 mouse myoblasts and MC-3T3 mouse pre-osteoblasts were also cultured on the scaffolds according to the protocols above. In the case of MC-3T3 cells, the DMEM was replaced with Minimum Essential Medium (ME) (ThermoFisher™).

To prepare bread scaffolds for seeding, they were placed in 70% ethanol for 30 minutes in order to sterilize them and subsequently rinsed twice with PBS. Bread scaffolds were additionally soaked in complete media prior to seeding to encourage adherence. A droplet containing 1.0×10⁵ cells was then gently placed on top of each of scaffolds, which were contained in 12-well plates. The samples were placed in the incubator for 3-4 hours to allow the cells to adhere to the scaffolds. The culture media was exchanged every 48-72 hours. Cells were maintained on scaffolds for two weeks in a standard cell culture incubator.

Differentiation of C2C12 and MC-3T3 cells was also carried out. C2C12 differentiation was initiated after first allowing the cells to grow to confluence over a period of two weeks. At this point, cells were cultured in myogenic differentiation media (DMEM, 2% Horse Serum, 1% penicillin/streptomycin) for up to two weeks in order to stimulate cell fusion and myogenesis. MC-3T3 cells were differentiated following a similar protocol but with osteogenic differentiation media (MEM, 10% fetal bovine serum, 1% penicillin/streptomycin, 50 μg/mL ascorbic acid and 10 mM p-glycerophosphate) for up to four weeks.

### 3. Staining

Before staining, the scaffolds were fixed in 4% paraformaldehyde for 10-15 minutes. Following 3 rinses with a duration of 5 minutes each in PBS, the samples were stained using 200 μL of a DAPI solution (1:500 in PBS) for 15 minutes to label nuclei. In cases where C2C12 and MC-3T3 cells were cultured, after fixation with paraformaldehyde, the cells were permeabilized with Triton X-100. Phalloidin alexa fluor 488 (ThermoFisher™) stock solution (1:100 in PBS) was incubated on the samples for 20 min at room temperature to label actin. In cases of antibody staining, samples were first washed with an ice-cold wash buffer (PBS, 5% FBS, 0.05% sodium azide) and placed on ice. C2C12 myotubes were labeled by incubating with an MF-20 myosin heavy chain primary antibody at a 1:200 dilution (DSHB Hybridoma Product) for 30 min followed by a rat anti-mouse IgG secondary antibody conjugated to Alexa Fluor 488 at a 1:100 dilution for 30 min. Between each stain the sample was incubated with the wash buffer for 30 min and the entire process was carried out on ice. In cases where deposited fibronectin was labelled the process was similar to the above. However, samples were incubated with a primary anti-fibronectin antibody at a 1:200 dilution (Abcam) for 30 min, followed by a rabbit anti-mouse IgG secondary antibody conjugated to Alexa Fluor 546 at a 1:100 dilution for 30 min. After staining, all scaffolds were rinsed for 2 minutes with PBS. The scaffolds were then stained with a 0.2% congo Red solution for 15 minutes, which was followed by 5-10 washes with PBS prior to mounting and imaging.

### 4. Confocal Microscopy

Confocal images were obtained using an A1R high speed laser scanning confocal system on a TiE inverted optical microscope platform (Nikon™, Canada) with appropriate laser lines and filter sets. Images were analyzed using ImageJ™ open access software. Brightness and contrast adjustments were the only manipulations performed to images. The ImageJ™ software was also used to count the number of cells in different areas of the scaffolds. Image analysis was conducted for quantifying pore size and volume fraction by collecting confocal Z-stacks, applying a threshold to obtain binary images at each optical plane, denoising and image quantification of pore area and volume.

### 5. Scanning Electron Microscopy

The preparation of the samples for SEM began with a fixation in paraformaldehyde. This was followed by a dehydration through successive washes of ethanol with increasing concentration (35%-99%). The samples were dried using a critical point dryer and gold-coated at a current of 15 mA for three minutes with a Hitachi™ E-1010 ion sputter device. SEM images were acquired at a voltage of 2.00 kV on a JEOL JSM-7500F FESEM. In the case of scaffolds cultured with MC-3T3 cells, energy-dispersive spectroscopy (EDS) was performed on three different areas of each scaffold surface and analyzed for mineral aggregates.

### 6. Cell Viability Assay

Cell viability was assessed with the Alamar blue assay (Invitrogen™). Cells were seeded onto scaffolds and assessed after 1 and 13 days in culture. In each case, samples were incubated with 10% (v/v) Alamar blue solution standard culture media for 2 hours in an incubator. Following incubation, the fluorescence was measured in a microplate reader at 570 nM against reference wavelength at 600 nM. The results are expressed in arbitrary units (AU) and normalized against the initial reads after 1 day in culture of the respective cell types.

### 7. Glutathione Assay

A Glutathione Assay (Cayman™ Chem) was conducted to evaluate the abundance of antioxidants within cells following incubation. Following two weeks of incubation the NIH3T3, C2C12 and MC-3T3 cultures were evaluated for glutathione content according to the manufacturer's guidelines. In brief, both 2D cell culture and 3D bread samples were collected and lysed in 2-(N-morpholino)ethanesulfonic acid (MES) buffer and centrifuged at 10,000×g for 15 minutes and then deproteinated with metaphosphoric acid (MPA). The resulting lysates were quantified against a standard curve as described by the supplier and normalized against the protein content of each sample by bradford assay.

### 8. Lactate Dehydrogenase Cytotoxicity Assay

Cytotoxicity was evaluated using the CyQuant™ LDH Cytotoxicity Assay (ThermoFisher™) to evaluate cell health. Samples of NIH3T3, C2C12 and MC-3T3 cells were incubated for two weeks in culture as described previously and compared against 3D TCP controls. Fractions referred to as “Spontaneous” (what is released in culture), and “Maximum” (the maximal value following lysis) were both collected to provide a %-Cytotoxicity value as described by the manufacturer. Results are expressed as the difference between the spontaneous and max reported values comparing the 2D TCP and 3D bread experimental conditions.

### 9. Mechanical Testing

The Young's modulus of the scaffolds was determined by compressing the material to a maximum 20% strain, at a rate of 3 mm/min, using a custom-built mechanical tester controlled with LabVIEW™ software. The force-compression curves were converted to stress-strain curves and the slope of the linear regime between 10-20% compression was fit to extract the Young's modulus.

### 10. Statistics

For the comparison of time series data, a one-way ANOVA™ with Tukey's post-hoc analysis was used to determine the statistical difference between sample populations. To compare between two distinct populations, a student's t-test was employed. In all cases alpha=0.05. Where indicated, all values are presented as the mean±standard deviation. Analysis and statistical tests were conducted using the OriginLab™ software package.

### Example 1: Preparing Sterile Bread-Derived Scaffolds

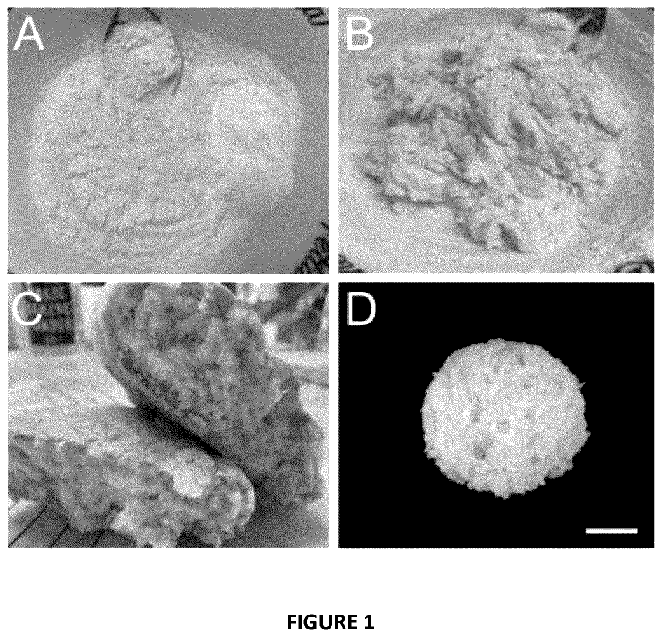

Scaffolds were prepared as described in the method section. As an initial step, dry ingredients were combined, followed by mixing in warm water and kneading (FIG. 1A, B). After baking, the internal part (crumb) of each loaf was characterized by a network of material which possesses variability in its porosity (FIG. 1C, D). To prepare the bread as a scaffold for supporting a cell culture, a 6 mm biopsy punch was used to extract a cylinder of material from the internal portion of the loaf, also known as the crumb. The cylinder was then sliced with a scalpel to create approximately 2.5 mm thick, 6 mm diameter circular pieces of material (FIG. 1D).

### Example 2: Mechanical and Structural Stability of Bread-Derived Scaffolds Overtime in Culture Conditions

This example examines cell proliferation and infiltration of bread crumb over the course of two weeks in culture. The results below show that the BB and cross-linked BB (xBB) scaffolds were stable over time in cell culture conditions and media.

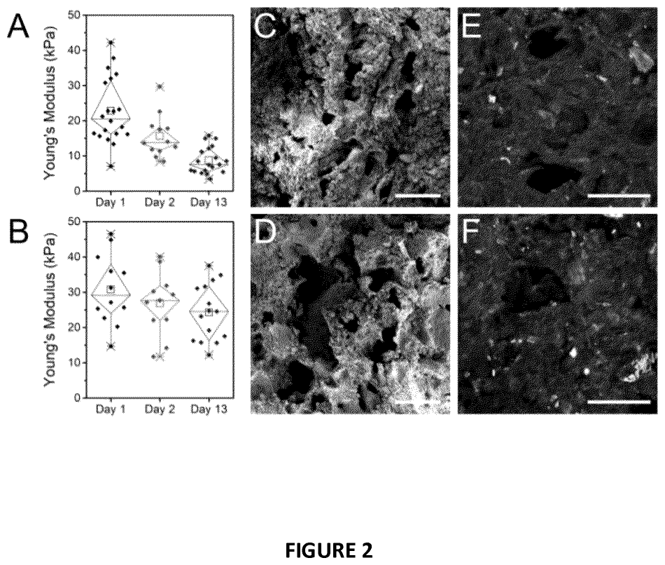

The baked bread (BB) scaffolds were continuously and completely submerged in cell culture media at 37 degrees Celsius for the entire length of time. Due to concerns that the native structure of the scaffold may begin to soften significantly and/or decompose over this time course, scaffolds cross-linked with glutaraldehyde (GA) were prepared to create a more stable structure. The mechanical properties of BB and xBB scaffolds were then measured after initially submerging in cell culture media (Day 1), 24 hr (Day 2) and 288 hr (Day 13) in culture media at 37 degrees Celsius with no mammalian cells (FIG. 2A, B). The results demonstrate that initially, there is no statistically significant difference in Young's modulus between the BB and xBB scaffolds due to the large variability (22.8±9.3 kPa and 30.8±9.9 p=0.06716). However, there is a difference in mechanical properties of the BB and xBB scaffolds as a function of time. By Day 13, BB scaffolds are observed to soften to 8.8±3.8 kPa (p=4.16854×10⁻⁶) compared to their state on Day 1. In contrast, the xBB scaffolds do not soften significantly by Day 13, maintaining a value of 24.2±8.1 kPa (p=0.27115). Although there is a slight downward trend in the xBB scaffolds, it is clearly not as significant as the BB scaffolds. Furthermore, by Day 13, the BB scaffolds are also softer than their xBB counterparts (p=2.32518×10⁻⁶).

Regardless, in both cases the BB and xBB scaffolds maintain their highly porous morphology and structure after immersion in cell culture media as evidenced by both SEM and confocal imaging (FIG. 2C-F). SEM and confocal imaging reveal that the pore sizes observed in the material can vary significantly over the range of micrometers to millimeters.

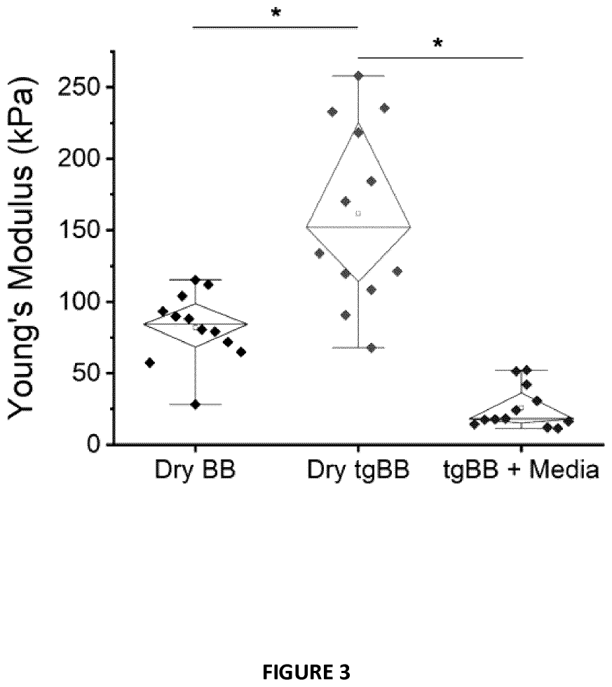

Transglutaminase (TG) is an alternative cross-linking agent that is compatible for use in food processing. To validate the use of TG for preparing food, the enzyme was added to the original formulation at a concentration of 1% (w/w) prior to baking. Once prepared, the crosslinked scaffolds (tgBB) generally appeared more hydrated and robust. Mechanical testing confirms that the addition of TG significantly (p=5.32719×10⁻⁴) increased the Young's modulus of the scaffold in its dry form (161.7±18.3 kPa, n=12) compared to the dry BB scaffold (82.1±7.1 kPa, n=12). (FIG. 3). Notably, once in culture medium the tgBB scaffolds (n=12) did soften but subsequently reached a stiffness of 25.6±4.3 kPa, consistent with the mechanical performance of the xBB scaffolds over time. The mechanical properties of the tgBB scaffolds did not differ statistically from the xBB scaffolds (FIG. 2B) throughout their time course (p>0.8).

### Example 3: Porosity and Pore Interconnectivity

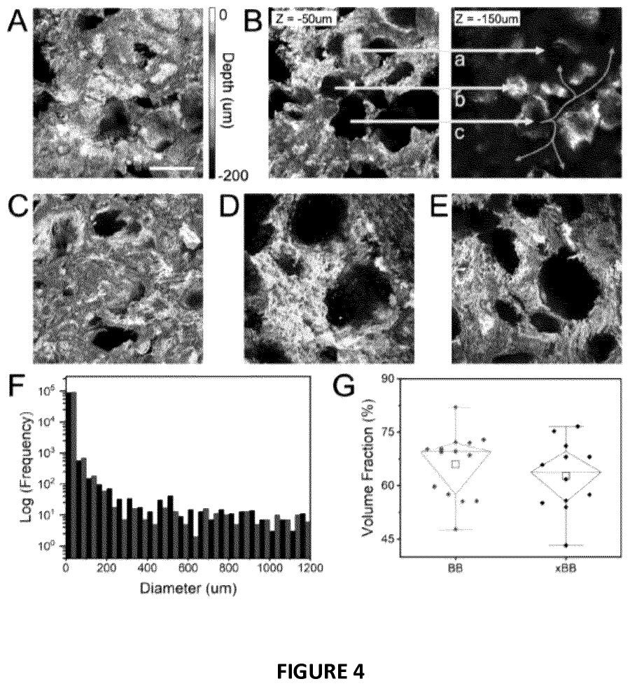

To further characterize the BB and xBB scaffolds, 1.3×1.3 mm confocal volumes were analyzed to quantify the porosity of the scaffolds. Depth coded confocal image stacks of the BB scaffold reveals a highly complex structure with a number of shallow pits and large pores which extend through the entire imaging volume (FIG. 4A).

The pore structure of the BB and xBB scaffolds was largely composed of individual isolated pores and surface pits, as well as networks of interconnected pores underneath the outer surface (FIG. 4B). Confocal optical sections in a representative BB scaffold acquired 50 μm below the outer surface reveal the presence of both relatively flat continuous surfaces, as well as cross sections through individual pores (FIG. 4B). The left image (FIG. 4B) reveals solid surfaces as well as open pores. A representative surface and two pores are identified by arrows a, b and c respectively (yellow). The arrows then point to the same region in the scaffold 150 μm below the surface (right image). Arrow “a” reveals how the solid surface in the left image covers a hollow void beneath (right image). Arrow “b” reveals the bottom surface (right image) of the pore identified in the left image. This particular pore is more accurately described as a pit and is isolated from the underlying open network. Finally, arrow “c” reveals how the bottom portion of a pore in the left image is actually open to an extensive interconnected volume of open space in the right image (green arrows).

A depth coded confocal image of an xBB scaffold is also shown for comparison (FIG. 4C). Notably, pore sizes were observed to vary dramatically over the surface of the scaffolds. Representative images of BB (FIG. 4D) and xBB (FIG. 4E) are also presented which demonstrate the presence of very large (300-500 μm diameter) pores which can be routinely observed in these scaffolds.

FIG. 4F shows a range of pore sizes can be extracted from confocal images of BB (red) and xBB (black) scaffolds. As depicted in FIG. 4G, BB (left) and xBB (right) scaffolds possess statistically indistinct volume fractions of 66.0±2.5% and 62.7±2.9% respectively (p=0.774565).

### Example 4: Cells can Infiltrate Deeply into the Scaffolds

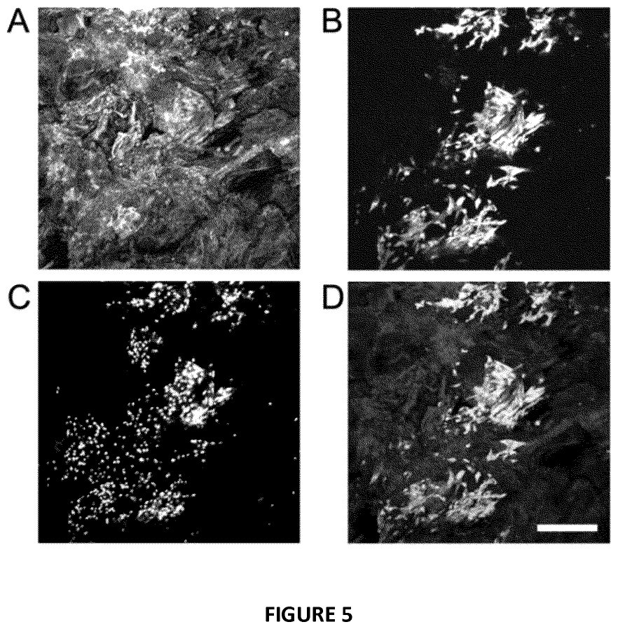

In order to assess the ability of the cells to penetrate into the scaffolds, they were cross sectioned as described previously (Hickey et al., 2018, Customizing the shape and microenvironment biochemistry of biocompatible macroscopic plant-derived cellulose scaffolds”, 4, 11, 3726-3736). Briefly, on Day 13, a 6 mm diameter, 2.5 mm thick xBB cylindrical scaffold was cut longitudinally with a microtome blade to produce two half cylinders. Subsequently, the cut side of the half cylinder could then be washed, fixed and prepared for imaging as described above. The results reveal that cells are indeed able to infiltrate into the deeper portions of the scaffold (FIG. 4 and FIG. 5). However, as expected the cell density by Day 13 inside the scaffold is much lower than the outer portion. This phenomenon is commonly observed in many 3D biomaterial scaffolds as it takes time for the cells to migrate deeply within a scaffold and, due to diminished oxygen/nutrient diffusion, the metabolic activity of these cells may be altered compared to cells closer to the exterior portion of the scaffold. As well, it is difficult to rule out how many cells were sheared off the scaffold surface during the sectioning process. Nevertheless, cells were still clearly observed deeply within the scaffold.

### Example 5: Cell Growth Dynamics on BB and xBB Scaffolds

In this example, to assess cell proliferation, the scaffolds were subsequently seeded with NIH3T3 cells stably expressing green fluorescent protein (GFP).

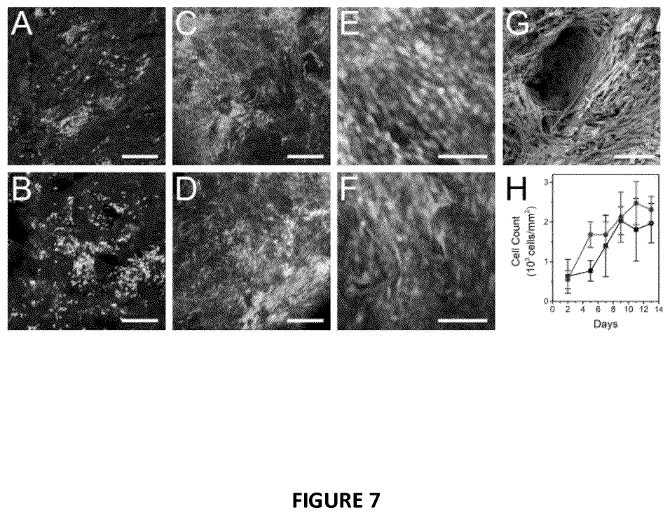

After seeding, scaffolds (n=3 at each time point) were subsequently imaged using confocal microscopy at Day 2, 5, 7, 9, 11 and 13. The presence of cells after two days reveals that they adhere to both formulations and tend to initially invade inside of the scaffold pores (FIG. 7A, B). By Day 13, the cells had clearly proliferated forming a high density of cells (FIG. 7C-F). SEM imaging on the BB scaffold reveals cells covering the surface as well as proliferating inside of the pores (FIG. 7G). For each scaffold formulation (n=3 per time point, per formulation), three randomly chosen 1.3×1.3 mm areas were imaged and the total number of cell nuclei were counted in each region and averaged (FIG. 7H). In both cases, the cell density on the scaffolds increases over time and eventually plateaus at about Day 9. The dynamics of cell growth do exhibit some variation during the experimental time course. Initially at Day 2, cell density on the BB and xBB scaffolds is the same (p=0.9) at 548±228 cells/mm² vs 628±428 cells/mm², respectively. However, at Day 5, the cell density on the BB scaffolds is significantly higher than the xBB scaffolds at 1682±323 cells/mm² vs 766±260 cell/mm², respectively (p=0.008). While there is some variation in the preceding time points, there are no statistically significant differences from Day 5 onwards. By Day 13, cell density on the BB and xBB scaffolds is the same at 2308±339 cell/mm² vs 1968±494 cells/mm², respectively (p=0.94569).

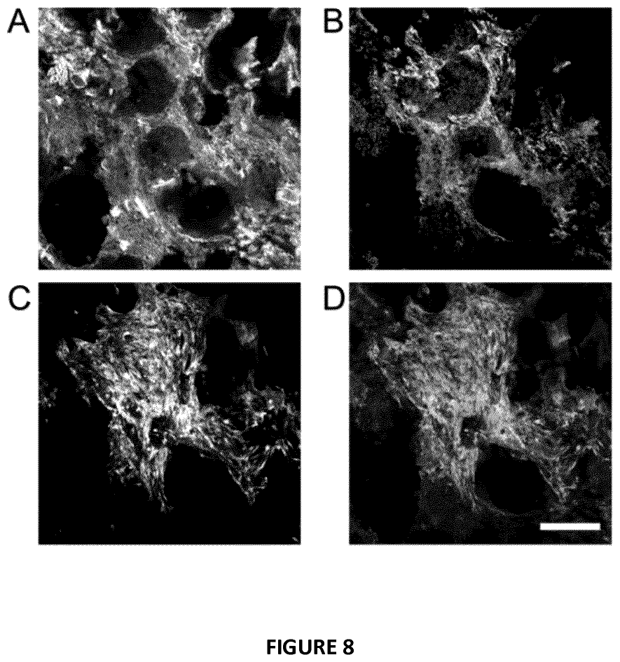

FIG. 8C and FIG. 8D shows that fibronectin deposition was largely localized to regions of significant cell density (all images are maximum Z-projections of confocal data, blue=scaffold, green=GFP cells, red=fibronectin). The images show that the cells deposited a cellular matrix, which evidences normal cellular functioning.

### Example 6: BB and xBB Scaffolds Support the Growth of Multiple Cell Types

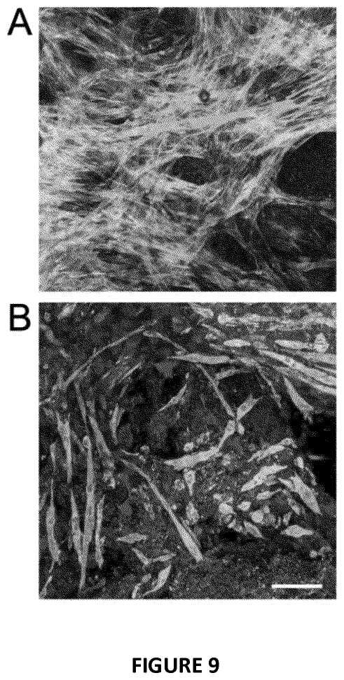

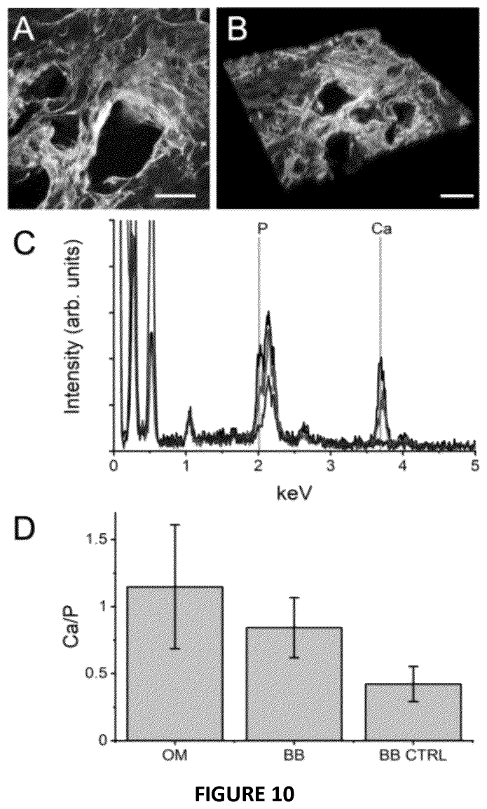

To demonstrate the utility of these scaffolds for multiple cell types, cultured C2C12 muscle myoblasts (FIG. 9) and MC-3T3 pre-osteoblasts (FIG. 10) were also cultured. These cell types were both chosen as they are established model cell types commonly employed in research for tissue engineered scaffolds. In addition to cell growth, these cell types are also useful as they can be differentiated into muscle myotubes or osteoblasts which can serve as a useful tool for assessing their behaviour on a novel scaffolding material compared to other common scaffolding types.

In the case of C2C12 cells, they were able to proliferate on both scaffold formulations in a manner consistent with the NIH3T3 cells. As they do not express GFP the actin cytoskeleton was stained in addition to the scaffold and nuclei (FIG. 9A). C2C12 myoblasts were observed to migrate across the surface of the scaffolds and exhibit well defined actin stress fibres. These myoblasts are also a lab model for muscle myogenesis in which the cells proliferate to confluence after which they can be serum starved to stimulate their fusion and differentiation into multinucleated myotubes. Without being limited by theory this is considered a key early step in muscle tissue growth and formation. The C2C12 cells were cultured on BB scaffolds (n=12) for two weeks prior to switching them into differentiation media for an additional one to two weeks of culture. When differentiated myotubes were observed on the surface of the multi-well plates (some even spontaneously contracting) the scaffolds were prepared for staining. To identify differentiated myotubes we stained with an antibody against myosin heavy chain, a key indicator of differentiation. Upon observation, myotubes were clearly observed on the scaffold surfaces consistent with more traditional substrates such as the standard plastic of tissue culture vessels (FIG. 9B).

In the case of the MC-3T3 pre-osteoblasts, the highly porous nature of the substrate is consistent with various other scaffolds encountered in bone tissue engineering. In these cases, pre-osteoblasts are differentiated into osteoblasts which can mineralize porous 3D microenvironments. Cells were first cultured for two weeks in proliferation media followed by switching into osteogenic media (OM) for an additional two weeks to differentiate. During differentiation with OM, or during biomaterial induced osteoinduction, this model cell line results in the formation of calcium and phosphorus rich mineral deposits on the underlying scaffold. In this case, after two weeks in proliferation media, cells were observed attached and proliferating on BB scaffolds in a manner consistent with the other cell types. As already noted, the scaffolds are highly porous and the cells are observed in the pores (FIG. 10A, B). After two weeks of proliferation, the cells were switched to OM for an additional two weeks. To assess the level of differentiation on the scaffolds, n=3 scaffolds were prepared and energy-dispersive spectroscopy (EDS) was performed at three randomly chosen sites on each scaffold. EDS spectra were acquired on differentiated cells, cells cultured for four weeks without OM and BB scaffolds alone (FIG. 10C). The EDS spectra clearly increased peaks occurring at 2.01 keV (phosphorus) and 3.69 keV (calcium) compared to the BB scaffold alone (FIG. 10C). Although the Ca/P ratio displays an increasing trend when comparing the BB scaffold alone and cells cultured with or without OM, the trend was not statistically significant (p>0.2 in all comparisons) (FIG. 10D). Therefore, while some degree of mineralization is taking place in the presence of cells (with and without OM), the levels are relatively low. However, further scaffold optimization may be carried out by those of skill in the art without undue experimentation to achieve a suitable level of mineralization.

In this study, our goal was to establish the possibility of utilizing highly available and accessible materials to create scaffolding capable of supporting mammalian cell growth. Taken together, data presented here demonstrates that these scaffolds are generally compatible with a multitude of cell types.

### Example 7: Metabolic Activity of Cells Proliferating on BB and xBB Scaffolds

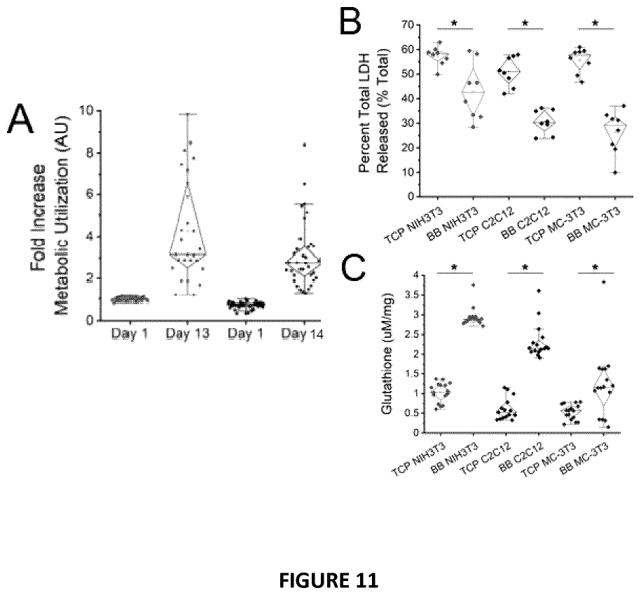

To assess the viability and metabolic activity of the cells at Day 1 and approximately 2 weeks after seeding (Day 13/14), an Alamar blue assay was employed. Such technique has been successfully used on 3D scaffolds and is described in Baino, F. et al., Processing methods for making porous bioactive glass-based scaffolds—A state-of-the-art review. Int. J. Appl. Ceram. Tech. 2019, 16, 1762-1796, which is incorporated herein by reference. Cell viability was determined by normalizing the measured values against the Day 1 controls to determine a relative fold-increase. As shown in FIG. 11A, for Day 1 and Day 13 BB scaffolds (red; leftmost data set) (n=32, n=27, respectively) cell viability increases significantly by a factor of approximately 3, on average 9 (p=7.50372×10⁻¹⁴). On both scaffold types, the increase in cell viability is significant compared to the initial state. While the BB scaffolds display variability relative to the xBB scaffolds (FIG. 11A black; rightmost data set), on average there appears to be more viable cells on these scaffolds (p=0.02143).

To assess the relative health of NIH3T3, C2C12, MC-3T3 cells when cultured on the scaffolds in comparison to traditional 2D tissue culture plastic, both a lactate dehydrogenase (LDH) and glutathione (GSH) assay were conducted. The LDH assay is a method for determining cytotoxicity and involves the measurement of certain enzymes released by damaged or necrotic cells. LDH is a cytoplasmic enzyme found in all cells and is released into the media when the plasma membrane is damaged or during cell death. Likewise, it has been well established that glutathione levels are a strong indicator of the ability of cells to detoxify and buffer against oxidative stress. When total glutathione levels are observed to decrease, this change can be measured to assess the degree of oxidative stress in a population of cells.

For the three cell types in question (NIH3T3, C2C12, MC-3T3) both LDH and GSH in populations of cells cultured on BB scaffolds were examined after two weeks in culture (FIG. 11B). In the case of LDH release, a statistically significant difference was observed on the 3D BB substrates compared to 2D substrates with comparable cell counts. For each cell type (n=8 in each condition), a significant decrease in LDH levels of about 20% was observed for all three cell lines (p=5.23×10⁻³, p=2.94481×10⁻⁶, p=1.27465×10⁻⁶ for the NIH3T3, C2C12 and MC-3T3 cells respectively). The results indicate that the cells on scaffolds are exhibiting less cytotoxicity; however, the observed levels on both tissue culture plastic and in the scaffolds are well within expected normal limits for many cell types.

Consistent with these observations, the GSH assay supports a similar conclusion. In this case, NIH3T3 (n=16 in both conditions), C2C12 (n=16 in both conditions), MC-3T3 were compared (n=15 and n=16, on tissue culture plastic and BB scaffolds respectively). In all cases, total GSH was observed to increase by a factor of two to four when cultured on BB scaffolds as opposed to tissue culture plastic (p=4.09462×10-20, p=1.54964×10⁻¹³, p=3.53×10⁻³ for NIH3T3, C2C12 and MC-3T3 cells respectively) (FIG. 11C). The increase in GSH is an indication that the cells are experiencing less oxidative stress when cultured on the BB scaffolds as opposed to the tissue culture plastic. While it may be possible that the BB scaffolds have sorptive properties, it is unlikely that this will account for such significant differences. Furthermore, experimental measures were taken to ensure type I errors (i.e., false positive) were not committed during analysis by deproteination of samples with MPA and ensuring experimental controls were performed on unseeded BB scaffolds. The levels of GSH are well within normal and expected limits for healthy cells.

The above description and examples are merely illustrative and additional embodiments and alternatives are included within the scope of the present disclosure.

1. A bread-derived scaffold biomaterial for supporting cells, the scaffold comprising a bread crumb and wherein the bread crumb comprises a three-dimensional porous structure to support the cells.

2. The bread-derived scaffold biomaterial of claim 1, wherein the bread crumb is a bread that is yeast-free.

3. The bread-derived scaffold biomaterial of claim 1, wherein the bread crumb is leavened with sodium bicarbonate.

4. The bread-derived scaffold biomaterial of claim 1, wherein the crumb comprises one or more gluten proteins.

5. The bread-derived scaffold biomaterial of claim 1, wherein the crumb comprises one or more non-gluten proteins.

6. The bread-derived scaffold biomaterial of claim 1, wherein the bread crumb is cross-linked with a cross-linking agent.

7. The bread-derived scaffold biomaterial of claim 6, wherein the cross-linking agent is glutaraldehyde or transglutaminase.

8. The bread-derived scaffold biomaterial of claim 1, wherein the crumb is for supporting the growth of any combination of cells that are selected from mammalian cells, fish cells, avian cells, reptile cells, amphibian cells, crustacean cells, plant cells, invertebrate cells, algae cells, bacteria cells, archaea cells or fungal cells.

9. The bread-derived scaffold biomaterial of claim 8, wherein the cells are fibroblast, satellite cell, myoblast, myocyte, smooth muscle cell, myofibroblast, myotube, cardiomyocytes, neutrophil, macrophages, lymphocytes, monocytes, platelets, pre-osteoblast, osteoblast, osteoclast, pre-adipocyte, adipocyte, periodontal ligament stem cells, fibrocytes, chondrocyte, tenocyte, keratinocytes, hepatocytes, neuron, neural precursor cells, dorsal root ganglion cells, glial cells, astrocytes, epithelial cells, endothelial cells, stem, mesenchymal stem or induced pluripotent stem cell or any combination thereof.

10. The bread derived scaffold of claim 1, wherein the crumb is for supporting cells that form a tissue that includes skeletal muscle, smooth muscle, cardiac muscle, bone, fat/adipose, kidney, liver, lung, skin, neural, vascular tissues or any combination thereof.

11. A tissue or cell-based product having a three-dimensional structure comprising the bread-derived scaffold biomaterial of claim 1, the bread-derived scaffold biomaterial having pores supporting a population of the cells.

12. The tissue or cell-based product of claim 11, wherein the product is a food product.

13. The tissue or cell-based product of claim 12, wherein the food product is a meat product.

14. The tissue or cell-based product of claim 13, wherein the population of cells comprise myocytes and/or adipocytes.

15. A process for producing a tissue or cell-based product comprising: growing cells on the bread-derived scaffold biomaterial of claim 1.

16. The process of claim 15, wherein the cells are grown on the scaffold in vitro.

17. The process of claim 15, wherein the cells are grown on the scaffold in vivo.

18. The process of claim 15, wherein the cells are grown on the scaffold in a vessel and wherein the scaffold serves as a microcarrier in the vessel.

19. (canceled)

20. (canceled)

21. A method for producing a bread-derived scaffold biomaterial of claim 1, comprising:
(i) preparing a dough comprising mixing a liquid, flour and a leavening agent;
(ii) baking the dough to produce a bread-product having an internal crumb; and
(iii) removing the internal crumb or a portion thereof from the bread-product to thereby provide the bread-derived scaffold biomaterial.

22. The method of claim 21, further comprising a step of sterilizing the bread-derived scaffold biomaterial.

23. The method of claim 21, further comprising cross-linking the bread-derived scaffold biomaterial with a cross-linking agent.

24. The method of claim 21, wherein the leavening agent is a chemical agent.

25. The method of claim 24, wherein the chemical agent is a salt of a bicarbonate ion.

26. The method of claim 22, wherein the liquid is water or milk.
